(19) United States
(12) Patent Application Publication
(10) Pub. No.: US 2025/0255698 A1
(43) Pub. Date: Aug. 14, 2025

(54) DENTAL IMPLANT FIXTURE, FIDUCIAL MARKER, SYSTEM, AND METHOD FOR PREPARATION AND USE

(71) Applicant: RIAD AND AMANDA ALMASRI REVOCABLE TRUST, Dallas, TX (US)

(72) Inventor: Riad Almasri, Dallas, TX (US)

(73) Assignee: RIAD AND AMANDA ALMASRI REVOCABLE TRUST, Dallas, TX (US)

(21) Appl. No.: 19/047,700

(22) Filed: Feb. 7, 2025

Related U.S. Application Data
(60) Provisional application No. 63/636,677, filed on Apr. 19, 2024.
(60) Provisional application No. 63/551,415, filed on Feb. 8, 2024.

(51) Int. Cl. A61C 8/00; A61B 90/00; G16H 20/40

(52) U.S. Cl. CPC A61C 8/0022; A61B 90/39; A61C 8/0013; A61C 8/0068; A61C 8/008; A61C 8/0089; G16H 20/40; A61B 2090/3937; A61B 2090/3966

(57) ABSTRACT

Described is an implant fixture having body formed as an elongated post which is fully threaded on a portion of the body and is partially threaded on a portion of the body, where the portion that is partially threaded forms a smooth outer side surface which can be polished and exposed outside the bone when implanted. Also described is a low profile unitary dental fiducial marker, drill bit for site preparation, and driver for fiducial marker placement, and a method for diagnostics and treatment planning of a dental surgical procedure using the fiducial marker, drill bit, and driver.

Dental implant systems include several components that work together to replace a missing tooth or teeth and restore oral function and aesthetics.

The primary components of a dental implant system typically include:
(1) an implant fixture.
The implant fixture is the component that is surgically placed into the maxilla or mandible (upper or lower jawbone). The implant fixture is preferably made of medical-grade titanium and is threaded to facilitate placement and osseointegration, i.e., its fusing with the jawbone. The implant fixture provides a stable foundation for the other components of the dental implant system. This component is often referred to as the “implant” when describing the components of an implant system;
(2) an abutment.
The abutment is a connector piece attached to the implant fixture once osseointegration has occurred. The abutment protrudes above the gumline and serves as a platform to which the prosthetic tooth or crown is attached. Abutments are available in various shapes and sizes to accommodate different clinical situations and esthetic requirements;
(3) a connector screw.
The connector screw secures the abutment to the implant fixture. The connector screw can be a small, precision-engineered screw that ensures a stable and secure connection between the abutment and implant fixture;
(4) a prosthetic tooth, crown, or bridge.
The prosthetic tooth or crown (or a plurality of teeth or crowns provided in a bridge) is custom-made from materials such as porcelain, ceramic, or zirconia, to closely mimic the color, shape, size, and general appearance of natural teeth. The prosthetic tooth or crown attaches to the abutment and functions as the visible part of the replacement tooth.

Together, these components provide a functional and natural-looking replacement for a missing tooth or teeth. Dental implant systems can vary in design and may include additional components or variations depending on the manufacturer and specific treatment plan. For example, an optional component used by dental health professionals is a healing cap or cover screw temporarily placed on top of the implant fixture after surgery to protect the area during the initial healing period. The healing cap or cover screw can be removed when the implant fixture is sufficiently integrated within the bone, and is replaced with the abutment.

Dental implant fixtures can vary in their dimensions and materials depending on the specific implant system, the manufacturer, and the individual patient's needs. However, implant fixtures are typically made from medical-grade titanium or titanium alloy due to the exceptional biocompatibility, strength, and corrosion resistance properties, which make it suitable for long-term use within the human body.

Implant fixtures can range from approximately 6 mm to 16 mm or more in length, depending on the location in the mouth and the amount of available bone. Dental implant fixtures are also provided in various diameters ranging from about 3 mm to 6 mm. The choice of diameter depends on the size of the edentulous (toothless) space and the quality of the surrounding bone.

Implant fixtures used in current dental implant practice are commonly provided with threads, i.e., they are configured as a screw-like (threaded) post and can have roughened surfaces to facilitate stability and osseointegration. Osseointegration is the process whereby the implant fixture becomes integrated with the surrounding bone tissue, and provides a stable foundation for the abutment and prosthetic affixed thereto. Certain implant fixtures comprising threads can be coated with, for example, a hydroxyapatite coating, or can undergo other surface treatments, such as acid etching or sandblasting, to roughen the surface and enhance osseointegration and accelerate the healing process.

These known threaded or surface-treated implant fixtures are fully threaded, i.e., they comprise threads that completely encircle the circumference of the implant fixture. However, even if coated, these fully threaded implant fixtures can be sub-optimal when the bone mass is insufficient to fully enclose all sides of the implant fixture, i.e., where the threads or coating or surface-treated area of the implant fixture is exposed outside the bone. For example, insufficient bone mass can present in patients with bone atrophy or bone resorption due to a variety of causes. Bone reconstruction and arch rehabilitation procedures are often recommended prior to performing implant surgery.

An alternative to prior bone reconstruction procedures is known as the “Palatal Approach technique” wherein a threaded implant fixture having at least a part of its outer surface comprising a rough surface. In this procedure, the threaded implant fixture is placed in the palatal area such that the rough surface is exposed to the palatal side. Bone reconstruction is then performed to cover the exposed rough surface, where the rough treated surface can facilitate osseointegration with the reconstructed bone.

What is needed is an implant fixture that is not fully threaded, having at least a portion of the implant fixture which has threads that do not completely encircle the implant fixture, and can provide secure and stable osseointegration and prevent or lessen soft tissue irritation and dehiscence, while eliminating or reducing the need for bone reconstruction in patients with bone atrophy or resorption.

Dental procedures such as emplacement of a dental implant can require preparation of the oral cavity and the surgical site prior to initiating the procedure. Proper position and orientation of an implant or other dental prosthetic can be an important consideration and can contribute to patient comfort and satisfaction. Part of the preparation for a dental implant or other dental prosthetic procedure can involve the use of a dental fiducial marker.

Dental fiducial markers are small, radiopaque objects placed within the oral cavity to serve as reference points for various dental procedures, particularly in the field of radiography and orthodontics/prosthodontics. Fiducial markers are designed to be visible on imaging scans such as X-rays, cone beam computed tomography (CBCT), magnetic resonance imaging (MRI), and digital photogrammetry, aiding in accurate diagnosis, treatment planning, and assessment of treatment outcomes.

In dentistry, fiducial markers are used in several applications such as assisting with the creation and placement of implants, dentures, and the like, based on the unique oral anatomy of the patient. They also serve to identify specific locations for guided surgery both on a virtual level as well as in the actual patient. The use of fiducial markers in dentistry allows practitioners to correctly identify placement position for oral prostheses to ensure the correct fit and helps guide surgeons and practitioners to the precise location where an oral procedure should be performed. Fiducial markers also make easier and more accurate the transition from a virtual model to a physical model or prosthesis.

The use of fiducial markers in dentistry has become increasingly prevalent due to advancements in imaging technology and the growing demand for precise and comprehensive dental care. Fiducial markers are particularly valuable in situations where precise measurement, alignment, or orientation of dental structures is required, such as in orthodontic treatment planning or dental implant placement.

Common types of dental fiducial markers include radiopaque spheres, cylinders, or crosses made from materials such as metal alloys, ceramic, or plastic-any material that can be readily imaged using the chosen imaging technology. Fiducial markers are strategically placed within the oral cavity, often affixed to bone or aligned with specific teeth or dental appliances, to provide reference points for accurate spatial localization and measurement during imaging procedures.

Overall, dental fiducial markers play a crucial role in modern dental practice by facilitating accurate diagnosis, treatment planning, and assessment, contributing to improved patient outcomes and satisfaction. As technology continues to advance, the use of fiducial markers is expected to become more integral to the practice of dentistry, further enhancing the precision and efficiency of dental care.

Originally, single-component (“unitary”) fiducial markers were considered inadequate for providing reliable and reproducible visibility when imaged. Fiducial markers comprising multiple components or parts (non-unitary fiducial markers), such as screws having separate, flat, radiopaque spacers were developed to improve visibility when imaged. However, these multiple-component fiducial markers are difficult to work with and can require multiple steps or more complex manipulation during placement.

A unitary fiducial marker has been marketed (ARCHTRACER™ Markers; Digital Arches, LLC, Mesa, AZ, USA). However, these ARCHTRACER fiducial markers comprise a flat flange as a base, the flat flange including a raised platform formed as a transverse ridge spanning one dimension (e.g., the width but not the length) of the flange.

A slotted screw head of the ARCHTRACER fiducial marker for receiving the driver is positioned atop the raised transverse platform and extends upward several mm from top face of the transverse platform, increasing the height at which the fiducial marker extends into the oral cavity. The position of the screw head above the raised transverse platform can cause discomfort to the patient and may irritate the tongue when coming into contact with the fiducial marker.

The ARCHTRACER fiducial marker can also require a specialized driver which engages both the screw head and the raised platform for turning and placing the fiducial marker within the bone of the patient. Moreover, the ARCHTRACER fiducial marker lacks any indicia for facilitating placement position and orientation of the fiducial marker.

The threaded shank of the screw forming part of the ARCHTRACER fiducial marker, which extends from a bottom face of the flange base, has a cylindrical body having a constant, uniform width of about 2 mm along its length, the cylindrical configuration continuing until the bottom end of the shank which is tapered to a point. The cylindrical-shaped body therefore comprises about 75% to 95% of the length of the screw shank, and the tapered end forms only about 5% to about 25% of the length of the screw shank. This screw shank configuration for the ARCHTRACER fiducial marker can result in the screw shank breaking or bending during placement, and decreases its stability in softer bone, such as “type 3” and “type 4” bone.

What is needed is:
a unitary fiducial marker that has a lower profile than the ARCHTRACER fiducial marker, and which is configured for easy, stable placement in hard or soft bone, while providing more comfort and less irritation for the patient once the fiducial marker is affixed in position, namely a dental fiducial marker comprising a flange base, where the flange base is without or does not include a raised transverse platform, and where the fiducial marker includes a threaded screw shank that is tapered, and not substantially cylindrical, along most of its length, wherein the flange base includes a slotted screw head or an unslotted bolt head formed or disposed centrally on its top surface. To facilitate placement and orientation of the fiducial marker, asymmetric indicia (e.g., a marking, lettering, or a design) can be provided (e.g., disposed, printed, embossed or debossed) on a top face of the flange base (when a slotted screw head is used) or on a top face of the bolt head;
a bone drill bit configured to form a starter or “pilot” hole at the position that the fiducial marker is intended to be placed, where the bone drill bit includes a peripheral housing surrounding the tip of the bone drill bit to gauge depth of the pilot hole and serving as a tissue marker;
a driver configured to engage the slotted screw head or unslotted bolt head of the fiducial marker having the needed features; and
a fiducial marker system or kit comprising a fiducial marker with asymmetric indicia, drill bit, and driver having the needed features.

These features, and the advantageous results arising therefrom, are provided by the invention described and shown herein.

### BRIEF SUMMARY OF THE INVENTION

The subject invention pertains to an implant fixture, wherein the implant fixture is an elongated post or rod forming a body of the implant fixture, wherein the body is configured to be partially threaded on its outer side surface and having at least a portion of the outer side surface configured to be polished or smooth. A portion of an implant fixture of the subject invention is fully threaded by having threads that completely encircle the circumference of the implant fixture at a proximal end. At a distal end of an implant fixture of the subject invention, the implant fixture is not fully threaded, i.e., it is partially threaded, being provided with threads partially encircling a circumference of the implant fixture. Preferably, the partially threaded portion has threads that encircle at least about one half of the outer side surface, leaving a portion of the outer side surface that has an unthreaded or smooth outer side surface. Preferably, this smooth portion of the outer side surface can be polished.

In one embodiment, the threaded portion of the implant fixture of the invention can be coated, e.g., by application of hydroxyapatite coating.

An implant fixture of the subject invention is preferably used when performing a Palatal Approach technique or whenever a patient has a lingual or buccal concavity defect.

The subject invention further concerns a dental implant system comprising a novel implant fixture as described, having a post portion which is partially threaded and partially polished or smooth, and comprising a conventional abutment, connecting screw, and prosthetic tooth.

The system can also optionally include a healing cap for temporary placement over the implanted implant fixture, wherein the healing cap is subsequently removed and replaced with the abutment when osseointegration is sufficiently completed.

In use, the polished or smooth surface is left exposed, outside the bone, and does not require subsequent bone reconstruction to cover the exposed polished or smooth surface. Accordingly, the steps of a method according to the subject invention include:
a) providing an implant fixture which is partially threaded and partially comprises a polished or smooth surface, as described herein;
b) placing the implant fixture within the jawbone of a patient in need of a dental implant at a depth consistent with placement of an implant fixture in a conventional implant procedure, such that the threaded surface is oriented within the bone and the polished or smooth surface is oriented such that it is exposed outside the bone;
c) affixing an abutment to the implant fixture; and
d) affixing a prosthetic tooth or plurality of teeth (a bridge) to the abutment.

The above method can include the further step of temporarily affixing a healing cap to the implant fixture prior to affixing the abutment to the implant fixture. In addition, a method according to the subject invention can also optionally include surgical placement or replacement of periodontal tissue over the exposed polished or smooth face of the implant fixture. Advantageously, a method of the subject invention does not require bone reconstruction.

This novel configuration for an implant fixture can advantageously and unexpectedly provide secure osseointegration and can prevent or lessen soft tissue irritation and dehiscence.

Preferably, a method of the invention is performed in a patient undergoing a Palatal Approach technique or whenever the patient has a mandibular lingual or buccal concavity defect. Accordingly, instead of having the threads exposed to the soft tissue (e.g., periodontal tissue) or using bone graft, the polished surface will be the only exposed part of the implant fixture, which can yield a higher success rate.

The subject invention further concerns a novel, comparatively low profile unitary dental fiducial marker, useful and advantageous for accurate diagnosis, treatment planning, and assessment of treatment outcomes in dental procedures, such as dental implants.

The invention also concerns a bone drill bit for use in placement of a fiducial marker, the drill bit having advantageous features that facilitate accurate and precise drilling depth and the subsequent visualization of the pilot hole by marking of the surrounding tissue.

The invention further concerns a driver configured to engage a fiducial marker of the invention for emplacement of the fiducial marker.

The invention encompasses a system comprising a fiducial marker, bone drill bit, and driver of the invention, and can include a kit comprising at least two or more components of the system.

A dental fiducial marker of the subject invention is an integrally formed, or single-unit (hereinafter “unitary”), fiducial marker comprising a slotted screw head or an unslotted bolt head, a substantially flat, geometrically shaped base flange supporting the screw head or bolt head, and a threaded, conical screw shank. The unitary dental fiducial marker can further comprise at least one indicia disposed on a top face of the flange base or the bolt head for recognition of positional orientation and alignment of the fiducial marker for imaging purposes in a dental procedure, such as dental implantation.

In a preferred embodiment, the geometrically shaped base is substantially flat, which means that the flange is about 1-4 mm in thickness, having a top face and a bottom face, and is formed having a plurality of sides of equal length, preferably being shaped as a square, having four sides, each about 6 mm in length. The geometry provides for the flat flange to form a diamond shape when the sides are oriented at a 45-degree angle between the vertical and horizontal axes of the longitudinal axis of the fiducial marker. The slotted screw head or unslotted bolt head is about 1-3 mm in height extending upwardly from the top face of the flat base flange, and is also square as viewed from its top face. The sides of the square screw head are preferably oriented at 45-degrees relative to the sides of the flat flange.

In an embodiment employing a slotted screw head, said screw head is slotted for receiving a matingly configured driver, and is preferably cross-slotted (having two slots at 90-degrees relative to one another, forming the shape of a cross or an “X”), wherein the slots are positioned centrally from the sides, extending substantially across the entire width of the square screw head and directed or pointing toward the corners of the diamond-shaped flat flange. Also preferably, the slots formed within the screw head extend about half to three-quarters of the screw head height, i.e., the depths of the slots are preferably not as deep as the screw head is high, providing four equal cubic bosses extending upwardly from the screw head base. The bosses are integral with the screw head base and are therefore not completely independent from one another, being interconnected by virtue of the screw head base. The bosses and screw head base, together, form the whole screw head. The slotted screw head has a height of about 2 mm above the flat, top face of the flange base, and the two cross slots have a depth of about 1 mm to about 1.5 mm from the top face of the bosses, and extending downwardly into the screw head.

In an embodiment employing a non-slotted bolt head, the bolt head is typically a geometric shape, e.g., square/diamond-shape, pentagonal, hexagonal, heptagonal, octagonal, or the like, and is configured to matingly engage with a wrench or socket of a matingly configured driver. In a preferred embodiment, the bolt head has a four-sided, square-shaped perimeter, which is offset by 90° from the four-sided square-shaped perimeter base.

Positioned and disposed on a top face of the marker is a marking or indicia. For example, in an embodiment employing a non-slotted bolt head, the marking or indicia can be disposed on a top, outward-facing surface of the bolt head. In an embodiment comprising a slotted screw head, the marking or indicia can be disposed on the flat flange, between the diamond point of the flat flange and one side of the screw. The entire fiducial marker, including the indicia disposed thereon, can be radiopaque. Preferably, only a single marking or indicia is provided on the top face of the flat flange or bolt head. The indicia can be presented as any random or ornamental design, code, numeral, letter or wording, to communicate positioning or orientation of the fiducial marker within the oral cavity, and is preferably asymmetric to facilitate the determination of its orientation and positioning. It would be understood that the indicia or marking can be formed by being printed, applied as a paint, engraved, stamped, embossed or debossed.

Extending from the bottom face of the flat flange is the screw shank or screw body, which is threaded and conical in shape, having its widest dimension at the interface with the bottom face of the flat flange base. The screw shank can comprise a variety of thread depths and pitches. Preferably, the screw shank of a fiducial marker of the invention is conical and tapered to a point at its distal end, forming a tap-screw configuration to facilitate placement. Conventional fiducial markers have threaded screw shanks having a length of about 5 mm to about 8 mm.

Each component of the fiducial marker of the invention—the screw or bolt head, flat flange base, and threaded, conical screw shank, are integrally fashioned and are unitary in that the fiducial marker is formed, machined, molded, 3-D printed, or otherwise manufactured as a single unit, and are not formed as separable components or parts of the fiducial marker.

The subject invention further includes a method for accurate diagnostics and treatment planning of a dental surgical procedure performed on a patient employing a fiducial marker described herein.

The method of the invention comprises:
providing a dental fiducial marker of the invention, i.e., an integrally formed, unitary fiducial marker comprising a slotted screw head or unslotted bolt head, a flat, geometrically shaped base flange, a threaded conical screw shank or screw body and, optionally, a radiopaque indica disposed on the top face of the flat flange base or bolt head;
implanting said dental fiducial marker at a predetermined position in the patient's mouth or dental arch; and
imaging the patient's mouth or dental arch to assess position, alignment, and relationships of dental structures in the patient.

A driver according to the invention includes a cylindrical shaft having a first, drill engagement end and a second, screw-head (to engage a slotted screw head) or bolt-head (to engage a non-slotted bolt head) engagement end. The first end of the driver shaft can be configured to detachably engage with a conventional dental drill and drill handle, wherein the drill provides rotational force to turn the driver. The second, and distal screw-head or bolt-head end of the driver shaft, comprises a driver “head” having a peripheral housing configured to engage the outer sides of the screw head or bolt head. In an embodiment comprising a slotted screw-head engagement end, a cross-shaped embossed blade can be formed and recessed within a cavity of the housing for engaging the cross-slotted screw head of the fiducial marker invention as described herein.

A bone drill bit of the invention comprises a cylindrical shaft having a first, drill engagement end and a second, drilling tip for forming a pilot hole in bone. The first end of the drill bit shaft can be configured to detachably engage with a conventional dental drill and drill handle, wherein the drill provides rotational force to turn the drill bit. The second, distal end of the drill bit is formed as a pointed, apical tip to facilitate drilling into bone. Advantageously, the drill bit of the invention further comprises a circular, disc-shaped flange affixed perpendicularly to the shaft, proximal to the second, distal drill end. The disc-shaped flange includes a sharp outer edge on its distal face (facing the drilling end) and can effect a circular cut or score into soft tissue overlying the bone and around the pilot hole. The flange, which can be affixed to or integrally formed with the drill bit shaft, is positioned such that its leading edge is a specific, fixed distance, preferably about 3 mm, from the tip, thereby serving to indicate and gauge depth of the drill bit tip, i.e., identify when the tip is about 3 mm, into the bone. The circular cut formed in the overlying tissue can mark the area surrounding the pilot hole and facilitate identifying the location of the small pilot hole, and can be used to remove the tissue around the pilot hole.

The drill bit, fiducial marker, and driver, as described herein, can be used together as a system for placement of the fiducial marker in a dental procedure, e.g., performing a dental implant procedure. The system comprising the described drill bit, fiducial marker, and driver, can be employed as an aid in accurate diagnosis, treatment planning, and treatment outcome assessment.

The system comprising a drill bit, fiducial marker, and driver, as described, can be packaged and sold as a kit for performing a dental procedure utilizing dental fiducial markers for diagnosis, treatment planning, and assessment of treatment outcomes.

In use, the system can be employed in a method comprising the steps of:
detachably attaching into a conventional dental drill, a bone drill bit comprising a perpendicularly disposed disc flange according to the invention;
drilling a pilot hole into a bone of the oral cavity of a patient at a position where a fiducial marker is to be placed, and concurrently marking the site of the pilot hole with the perpendicular disc flange;
inserting a fiducial marker as described into the pilot hole; and
driving the fiducial marker into the pilot hole to a desired depth using the described driver.

These and other features of the invention can be readily understood and practiced by referring to the description herein and the accompanying drawings set out and included in this specification.

### BRIEF DESCRIPTION OF THE DRAWINGS

FIG. 1 is a side view of an implant fixture according to an embodiment of the invention, illustrating a post having a portion thereof threaded and a portion thereof unthreaded, wherein the unthreaded portion is smooth or polished.

FIG. 2 is a perspective view of an implant fixture according to the subject invention, showing the implant fixture oriented in relation to maxillary bone of a dental implant patient.

FIG. 3 is a perspective view of an implant fixture according to the subject invention, showing the implant fixture oriented in relation to mandibular bone of a dental implant patient.

FIG. 4 is a distal end view of an implant fixture according to an embodiment of the subject invention, showing the polygonal opening for receiving a drill bit for placement, and illustrating the threaded portion and polished or smooth portion of the implant fixture with a round mark to point out the threaded surface from that view.

FIG. 5 is a front elevational view of an implant fixture according to an embodiment of the invention illustrating the features of the invention.

FIG. 6A shows a top-view of an embodiment of a dental fiducial marker of the subject invention, illustrating the “diamond” shape formed by a square, flat flange base, the relative orientation of the square bolt head upon the flange base, and the position of the indicia provided on an outer (upper) face of the square bolt head and the corner of the diamond-shaped flat flange.

FIG. 6B shows a side elevational view of an embodiment of a dental fiducial marker of the subject invention, illustrating the positional relation of the bolt head, flat flange base, and conical threaded shank of the fiducial marker.

FIG. 7 shows a side, sectional view of a driver which matingly engages with a bolt head of a fiducial marker of the invention.

FIG. 8 shows a side view of a drill bit according to the invention comprising a perpendicular disc flange having a sharp distal cutting rim useful as a tissue marker or tissue “punch.”

FIG. 9 illustrates a kit comprising at least one component each selected from a) a fiducial marker having a bolt head, b) a drill bit having a disc-shaped flange as a tissue punch, and c) a driver for a fiducial marker.

FIG. 10A shows a top-view of an embodiment of a dental fiducial marker of the subject invention, illustrating the “diamond” shape formed by the square, flat flange, the relative orientation of the square screw head, and cross-slots formed therein, and the position of the indicia provided between one side of the square screw head and the corner of the diamond-shaped flat flange.

FIG. 10B shows a side elevational view of an embodiment of a dental fiducial marker of the subject invention, illustrating the positional relation of the screw head, flat flange base, and conical threaded shank of the fiducial marker.

FIG. 11 shows a side, sectional view of a driver which matingly engages with a screw head of a fiducial marker of the invention.

FIG. 12 illustrates a kit comprising at least one component each selected from a) a fiducial marker, b) a drill bit having a disc-shaped flange as a tissue punch, and c) a driver for a fiducial marker.

### DETAILED DESCRIPTION OF THE INVENTION

An implant fixture according to the subject invention can be described in reference to the drawings attached hereto.

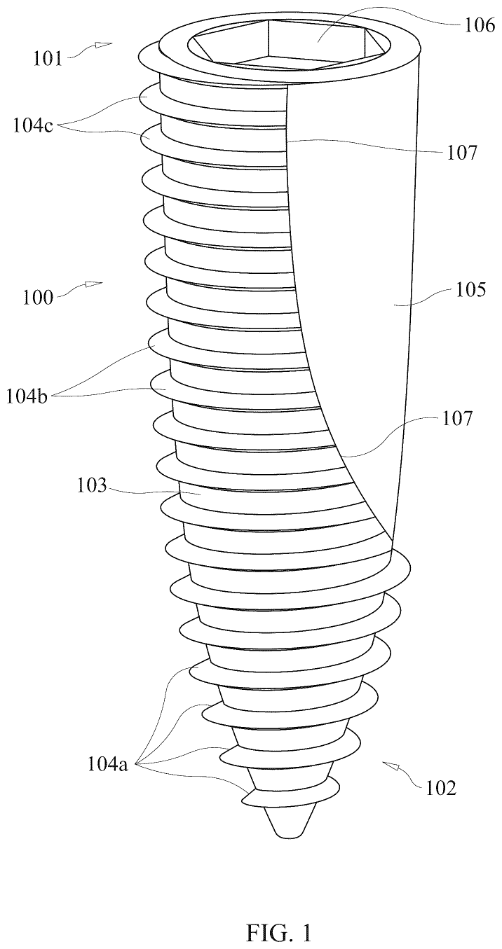

Referring to FIG. 1 is a side view of an implant fixture 100 according to an embodiment of the invention. As shown, the implant fixture 100 has a solid body portion 103 shaped as an elongated post or rod, wherein the body of the implant fixture has a first (distal) end 101, which can be substantially flat, and a second (proximal) end 102, which can be tapered. Implant fixtures can vary in dimension, and are typically about 6 mm to about 16 mm in length or height. A typical implant fixture is about 8 mm to about 12 mm in length or height. A standard width of an implant fixture is about 3 mm to about 6 mm (outer diameter) at its widest (distal) end, inclusive of threads, which can extend about 0.1-0.2 mm beyond the solid body 103 of the implant fixture 100.

A conventional implant fixture has a first, or distal, end 101 configured to function as a set screw, comprising a recess 106 that can receive and be tightened or loosened using a hex wrench or Allen wrench (not shown). It would be understood in the art, without deviating from the spirit of the invention, that other embodiments of the first end 101 can instead include alternative screw or screw head configurations. For example, first end 101 of implant fixture 100 can be slotted or recessed to matingly receive a flat, square, Phillips, or torx screwdriver or drill bit, or the like. A recessed first end which can engage and be tightened or loosened by a hex or Allen wrench is preferred for a dental implant fixture of the invention. This preferred configuration of the first end can facilitate receiving a standardized abutment or temporary healing cap placed on the distal end of the implant fixture in a conventional dental implant procedure.

Second end 102, also referred to as the proximal end or apical end, of an implant fixture according to the subject invention, preferably has an angled or tapered configuration, similar to a self-tapping wood or metal screw. It would be understood that alternative embodiments can comprise a flat or non-tapered second end, providing a barrel-shaped implant fixture or post. Second end 102 is preferably fully threaded, having threads 104a completely encircling the implant fixture body.

Preferably, these completely encircling threads 104a provided at second or proximal end 102 extend about one-quarter to about one-third the length or height of implant fixture 100. In one embodiment, the completely encircling treads extend distally only about 10-50% of the body 103 of the implant fixture 100. Preferably, the completely encircling threads extend distally about 2-6 mm, more preferably about 3-5 mm, and most preferably about 4 mm, from second end 102. These completely encircling threads can be coated with, for example, a hydroxyapatite coating, or can undergo other surface treatments, such as acid etching or sandblasting, to enhance osseointegration and accelerate the healing process.

The remaining 50-90% of the body of implant fixture 100 also comprises threads, but these threads on the remaining portion of the implant fixture body are formed such that they do not completely encircle the body of the implant fixture. These threads 104b and 104c are referred to herein as incomplete or truncated threads. Preferably, truncated threads 104b and 104c encircle only a portion of the body of implant fixture 100, specifically from about 50%-99% around the circumference of the implant fixture, depending on their position relative to first end 101. For example, truncated threads 104c encircle about 50% of the circumference of implant fixture 100. As the truncated threads continue proximally, they extend an increased distance around the circumference of implant fixture 100. Truncated threads 104b are shown as extending more than 50% around the circumference of implant fixture 100, and further proximally positioned truncated threads extend from 70-99% around the circumference of implant fixture 100, until reaching the position of the completely encircling threads 104a.

Preferably, this configuration is achieved by grinding down or removing the threads along a portion of implant fixture to provide a smooth or polished area 105. Polished or smooth area 105 provides about 50% of the surface area of implant fixture 100 at the first, distal end 101. Polished or smooth area 105 has an interface 107 with truncated threads 104b and 104c, which is curved, such that the polished or smooth area forms only about 1% or less of the surface area of implant fixture just above the completely encircling threads 104a. In other words, the surface area covered by polished or smooth area 105 is graduated, being a maximum of about 50% at first, distal end 101, and being less than 50% as the area extends proximally from first end 101.

Polished or smooth area 105 gradually decreases to form only about 1% of the surface area as it extends proximally along the length of implant fixture 100, and forms none or 0% of the surface area at the position where the completely encircling threads are provided.

Thus, in a preferred embodiment, the subject invention comprises an implant fixture having completely encircling threads on a portion of the implant fixture and truncated threads on a remaining portion of the implant fixture, wherein the truncated threads leave a smooth area along one side of the body of the implant fixture. The completely encircling threads extend distally from a proximal end of the implant fixture about 2-6 mm. The truncated threads reach about 1%-50% around the circumference of the body of the implant fixture, and are present from a distal end of the implant fixture and extend proximally to the completely encircling threads. The truncated threads provide or form a smooth area on one side of the implant fixture. This smooth area can be polished to remove any rough surface.

When used in a patient having bone atrophy, bone resorption, or other bone loss, where bone mass is insufficient, an implant fixture according to the subject invention, having a polished or smooth area along one side of the implant fixture, can be advantageous. The implant fixture of the subject invention can preferably be placed such that the polished or smooth area of the implant fixture is exposed on the outer surface of the bone. The exposed polished or smooth surface can lessen soft tissue irritation and dehiscence. The implant fixture of the subject invention having truncated threads on the remaining portion of the fixture can provide secure osseointegration such that bone reconstruction can be minimized or eliminated.

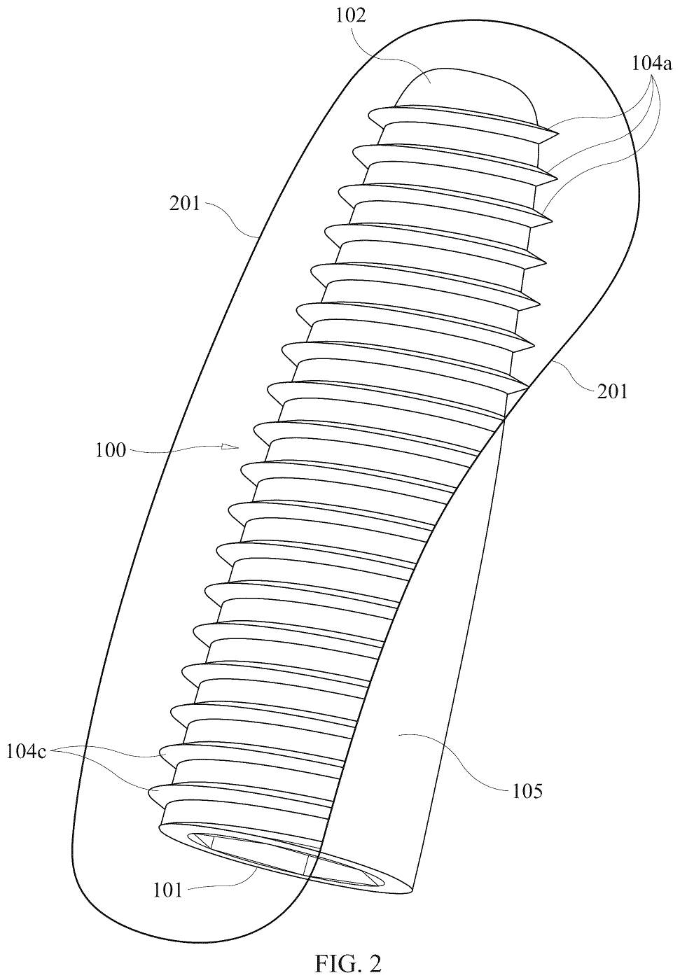

FIG. 2 is a perspective view of an implant fixture 100 according to the subject invention, showing a tapered embodiment of an implant fixture oriented in relation to maxillary bone 201 of a dental implant patient. Specifically, second apical (proximal) end 102 having threads 104a completely encircling the implant fixture is positioned within the bone, leaving first (distal) end 101 exposed to the occlusal side of the bone. Implant fixture 100 can preferably be positioned within the maxillary bone such that the partially threaded portion, having threads 104a not completely encircling the body of implant fixture 100, are oriented within the bone to enhance osseointegration, and smooth or polished portion 105 of implant fixture 100 is exposed outside the buccal or facial side of the bone 201. The smooth or polished surface can remain exposed outside the bone, without requiring further bone reconstruction to cover the smooth or polished portion.

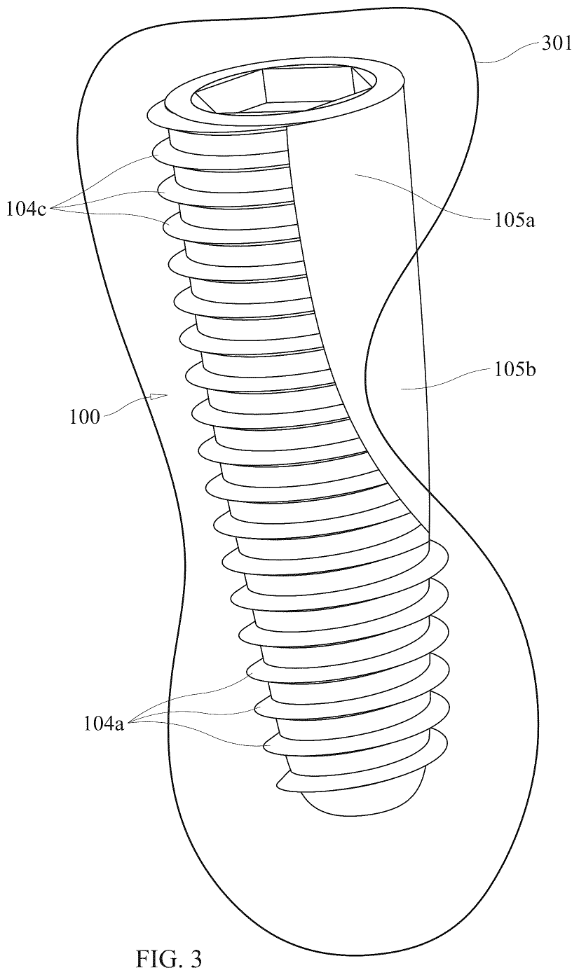

FIG. 3 is a perspective view of an implant fixture 100 according to the subject invention, showing a tapered embodiment of an implant fixture oriented in relation to mandibular bone 301 of a dental implant patient. Specifically, second apical (proximal) end 102 having threads 104a completely encircling the implant fixture is positioned within the bone, leaving first (distal) end 101 exposed to the occlusal side of the bone. Implant fixture 100 can preferably be positioned within the mandibular bone such that the partially threaded portion, having threads 104a not completely encircling the body of implant fixture 100, are oriented within the bone to enhance osseointegration. In this illustration, implant fixture 100 is shown to have a portion of smooth or polished portion 105b exposed outside the buccal or facial side of the bone 201, and a portion of smooth or polished portion 105b positioned within the bone. Smooth or polished surface portion 105b can remain exposed outside the bone, without requiring further bone reconstruction to cover the smooth or polished portion.

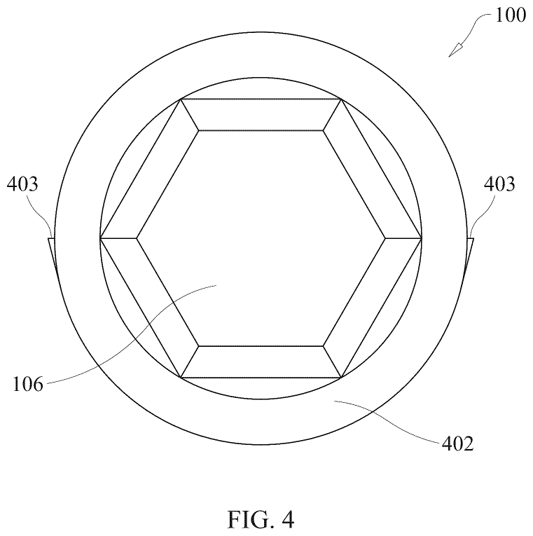

FIG. 4 is a distal end view of an implant fixture according to an embodiment of the subject invention, showing the polygonal opening 106, for receiving a drill bit, e.g., hex wrench fitting, for placement of the implant fixture, and illustrating the threaded portion 401 positioned within bone 403 and the smooth or polished portion and polished or smooth portion 402 of the implant fixture 100 exposed outside bone 403.

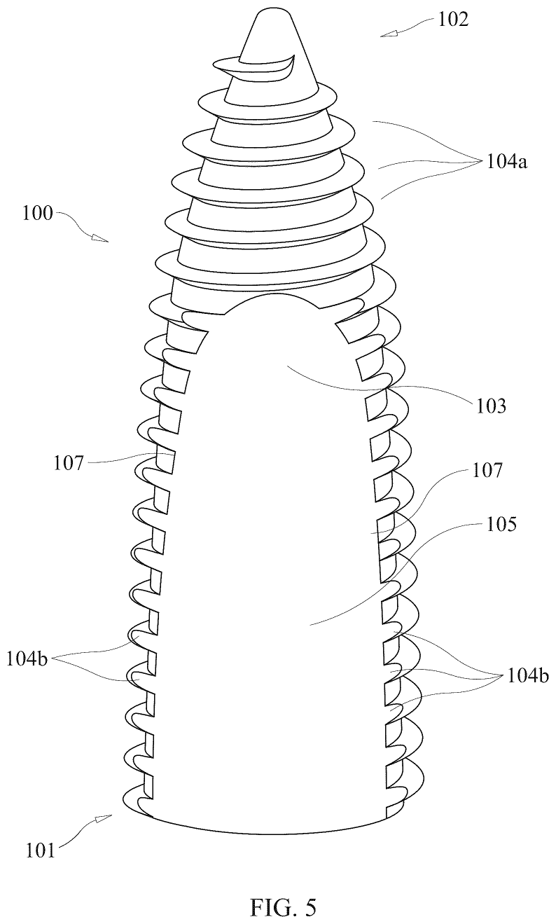

A front elevational view of an implant fixture of the invention is illustrated in FIG. 5. This view illustrates the smooth or polished portion on an outer side surface of a tapered embodiment of an implant fixture. Shown in this drawing is implant fixture 100 having a distal end 101 and a proximal end 102 as ends of implant fixture body 103.

Still referring to FIG. 5, at proximal end 102, the implant fixture is fully threaded, i.e., threads 104a are configured to completely encircle the circumference of the implant fixture. At distal end 101, threads 104b are formed on a portion of a side outer surface of the implant fixture, and partially encircle the implant fixture. Where the threads do not completely encircle the implant fixture body, a smooth or polished area 105 is formed on the outer side surface of the implant fixture. Threads 104b which do not completely encircle the implant fixture body border smooth or polished area 105 at interface 107. Interfaces 107 bordering smooth or polished portion 105 can form parallel lines or can be arched such that the interfaces on each side of the smooth or polished area adjoin toward the proximal end of the implant fixture.

The smooth or polished surface 105 can be formed by heating, molding or, preferably, grinding down and polishing the outer side surface of the implant fixture. The resulting smooth or polished area 105 is formed such that the outer side surface of the smooth or polished area is level with the internal crevasse formed by the threads.

Advantageously, periodontal tissue can re-grow or can be surgically replaced over the smooth or polished portion, wherein the smooth or polished area of the implant fixture can lessen soft tissue irritation and dehiscence. The partially threaded portion of the implant fixture, i.e., having threads that do not completely encircle the body or the implant fixture, does not diminish the osseointegration and can serve to secure the implant in place with substantially equal success as an implant fixture having threads completely encircling the entire implant fixture body. Moreover, an implant fixture configured as described herein for the subject invention can be preferred for use in a Palatal Approach technique where bone mass is diminished, and can be used without requiring bone reconstruction over the bone-diminished area.

The subject invention comprises a dental device, namely, a unitary fiducial marker and method for using the device to assess position, alignment, and relationships of dental structures in a patient which are useful in diagnosing and planning a dental procedure such as a surgical dental procedure, e.g., performing a dental implant procedure.

Recognized benefits of dental fiducial markers include:
Enhanced Precision: By providing clear reference points on imaging scans, dental fiducial markers enable dentists and orthodontists to accurately assess the position, alignment, and relationships of dental structures.
Improved Treatment Planning: Fiducial markers aid in the precise planning of dental treatments such as orthodontic tooth movement, dental implant placement, and surgical procedures by providing reliable landmarks for measurement and evaluation.
Reduced Radiation Exposure: With the aid of fiducial markers, dental professionals can minimize the need for repeat imaging scans, thereby reducing the overall radiation exposure to patients while ensuring diagnostic accuracy.
Facilitation of Communication: Dental fiducial markers serve as visual aids for communication between dental specialists, allowing for better interdisciplinary collaboration and coordination of treatment plans.

Dental photogrammetry is an advanced digital technique used in dentistry to precisely record the intraoral position of multiple dental implants. A dental fiducial marker is a specialized tool used in various dental applications, including dental photogrammetry. The precise configuration of dental fiducial markers can play a vital role in guiding imaging and placement of dental prosthetics (e.g., dentures, implants, and the like), in surgical procedures within the dental field.

Dental fiducial markers play a crucial role in implant dentistry, and can be utilized to aid in precise diagnostics, treatment planning, and surgical procedures. Specifically, dental fiducial markers are strategically positioned on the patient's denture or surgical guide, serving as reference points during cone beam computed tomography (CBCT) scans, intraoral radiographs, and other imaging modalities.

Radiopaque material used for making the fiducial markers can ensure clear visibility in X-ray images. Radiopaque material can include metal, e.g., lead, titanium, and the like, specialized ceramics or plastics which can facilitate imaging using conventional imaging techniques. This allows clinicians to accurately assess bone quality, anatomical structures, and implant sites. Accordingly, fiducial markers aid in creating surgical guides. These guides are customized based on CBCT data and incorporate the precise position of the markers. Surgeons use these guides during implant placement to ensure accurate angulation and depth.

During implant surgery, the markers help align the surgical guide with the patient's anatomy. This ensures that the implants are placed precisely according to the treatment plan. After implant placement, fiducial markers assist in follow-up assessments. Clinicians can track the healing process, evaluate osseointegration, and detect any complications.

In cases of multiple implants, fiducial markers aid in identifying specific implant sites during long-term follow-up visits. Fiducial markers serve as a quality control measure. Clinicians can verify the accuracy of implant placement by comparing the actual position with the planned position.

In summary, dental fiducial markers enhance precision, facilitate guided implant surgery, and contribute to successful outcomes in implant dentistry.

The subject invention provides a particularly useful dental fiducial marker configuration. A dental fiducial marker according to the invention is a unitary device, i.e., all components are integral with one another as a single-unit.

A dental fiducial marker device of the invention comprises a cross-slotted screw head, a flat, square or diamond-shaped flange base, and a conical, tapered and threaded screw shank. The flat flange base comprises radiopaque indicia placed on a top face of the flat flange base to provide an asymmetrical identifier on the otherwise symmetrical marker. This will help merge the files when designing the prosthesis for a more precise and accurate delivery. As in intra-oral scanning procedures, saliva and blood can get in the way of the images; this will help give the clinician preferred markers to merge images.

A fiducial marker of the invention can preferably be placed into a patient using a driver configured to engage with the square and cross-slotted screw head of the fiducial marker according to the invention.

Prior to placement of the fiducial marker into a patient, a preferred procedure can employ a drill bit to form a pilot hole into the bone as a guide for positioning the fiducial marker. A bone drill bit of the invention comprises a cylindrical shaft having a first, drill engagement end and a second, drilling tip for forming a pilot hole in bone.

The first end of the drill bit shaft can be configured to detachably engage with a conventional variable-speed dental drill and drill handle, wherein the drill provides rotational force to turn the drill bit.

The second, distal end of the drill bit is formed as a pointed, apical tip to facilitate drilling into bone. Advantageously, the drill bit of the invention further comprises a circular, disc-shaped flange affixed perpendicularly to the shaft, proximal to the second, distal drill end.

The disc-shaped flange includes a sharp outer edge on its distal face (facing the drilling end) to effect a circular cut or score into soft tissue overlying the bone and around the pilot hole. The disc-shaped flange, which can be referred to as a “tissue punch,” is about 2 mm in diameter, and is affixed to or preferably integrally formed with the drill bit shaft, and is positioned such that its leading edge is a specific, fixed distance, preferably about 3 mm, from the drill bit distal tip, thereby serving to indicate and gauge depth of the drill bit tip, i.e., identify when the tip is about 3 mm, into the bone. The circular cut or score formed in tissue overlying the bone can mark the area surrounding the pilot hole and facilitate identifying the location of the small pilot hole. In some instances, the cutting edge of the disc-shaped flange can remove the soft tissue around the pilot hole, thus serving as a tissue punch.

The drill bit, fiducial marker, and driver, as described herein, can be used together as a system for placement of the fiducial marker in a dental procedure, e.g., performing a dental implant procedure. The system comprising the described drill bit, fiducial marker, and driver, can be employed as an aid in accurate diagnosis, treatment planning, and treatment outcome assessment.

The system, comprising a drill bit, fiducial marker, and driver, as described, can be packaged and sold as a kit for performing a dental procedure utilizing dental fiducial markers for diagnosis, treatment planning, and assessment of treatment outcomes.

In use, the system can be employed in a method comprising the steps of:
detachably attaching into a conventional dental drill, a bone drill bit comprising a perpendicularly disposed disc flange according to the invention;
drilling a pilot hole into a bone of the oral cavity of a patient at a position where a fiducial marker is to be placed, and concurrently marking the site of the pilot hole with the perpendicular disc flange;
inserting a fiducial marker as described into the pilot hole; and
driving the fiducial marker into the pilot hole to a desired depth using the described driver.

In describing the device of the invention, reference is made to “top,” “superior,” “bottom,” “inferior,” and “side” or “lateral,” wherein “top” or “superior” refers to the direction toward the screw head, “bottom” or “inferior” refers to the direction toward the angled tip of the screw shank, and “side” or “sides” and “lateral” refer to a lateral aspect of the device.

An embodiment of a fiducial marker device of the invention is shown in the attached drawings and accompanying text provided in this specification. An embodiment of a fiducial marker driver as described herein is also shown in the accompanying drawings. A drill bit comprising a disc-shaped cutting flange is also shown in the accompanying drawings. These embodiments are exemplary only, and are by no means considered as limitations to the scope of the invention as described or its equivalents that are readily understood by a person of ordinary skill in the art.

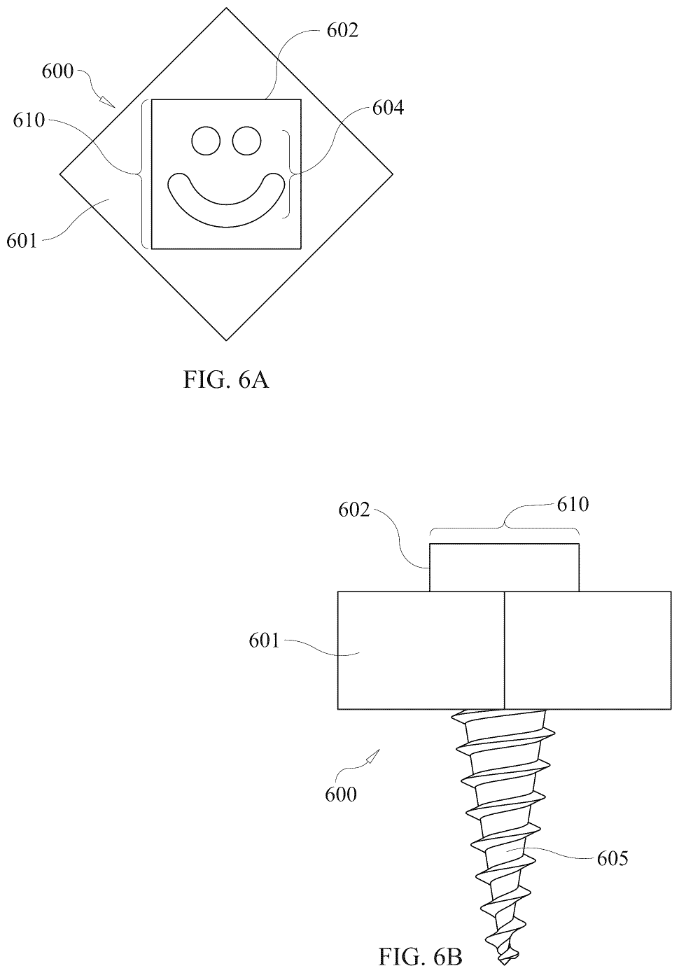

Referring to the drawings, FIG. 6A shows a top-view of an embodiment of a dental fiducial marker 600 according to the subject invention, illustrating a square, flat flange base 601 which is shown as being “diamond” shaped due to its orientation. The top face of flange base 601 includes, integral therewith, a geometrically shaped bolt head 610, shown here to be geometrically shaped as a square, having four sides. It would be understood that the bolt head can be variable in its geometric shape, and can be rectangular, pentagonal, hexagonal, octagonal or the like.

The flat flange can be about 4 mm to about 8 mm in length on each of its sides, and can be about 1 mm to about 4 mm in thickness. In a preferred embodiment, the flat flange base is about 6 mm in length on each side ad about 3 mm in thickness.

On a top face of the flat flange base 601 is formed a bolt head 610. Preferably, the bolt head 610 extends about 2 mm above the top face of the flat flange base and is square, measuring about 1 mm to about 4 mm on each side. Preferably the bolt head 610 measures about 4 mm×4 mm. The square bolt head is orientated such that the sides are positioned at a 45-degree angle relative to the sides of the flat flange base 601.

Disposed on a top face of bolt head 610 is an indicia 604, useful to ensure accurate and reproducible orientation of the fiducial marker. Indicia 604 is shown in FIG. 6A as a “smiley face” configuration, but can be any identifier, including any identifying shape or design, number or numbers, letter or letters, or the like. Indicia 604 can be radiopaque and comprises a single mark or identifier on the top face of the bolt head 610. Alternatively, the indicia can be positioned between one side face of the bolt head and a corner of the square or diamond-shaped, flat flange base. A preferred size of indicia covers an area having about 1 mm to about 3 mm radius.

On a bottom face of the flat flange base is disposed a screw shank, as illustrated in FIG. 6B. FIG. 6B depicts a side elevational view of an embodiment of a dental fiducial marker 600 illustrated in FIG. 6A, showing the positional and spatial relation of the integral flat flange base 601, bolt head 610, and threaded, tapered (conical) screw shank 605 extending from the bottom face of the flat flange base 601. Threaded screw shank 605 can be any needed length, typically from about 5 mm to about 8 mm in length, and is preferably about 6 mm in length. More preferably, threaded screw shank 605 is conical and tapered to a point at its distal end to form a tap-screw configuration for ease of placement. A preferred width of the tapered, conical screw shank 605 corresponds to the bolt head 610, and is preferably about 2 mm to 3 mm in width at its widest dimension, at the juncture with the bottom face of flange base 601, tapering down to a point at the bottom end of conical screw shank 605. Thread sizes (width, depth, and pitch) can vary and are accordance with conventional dental screws.

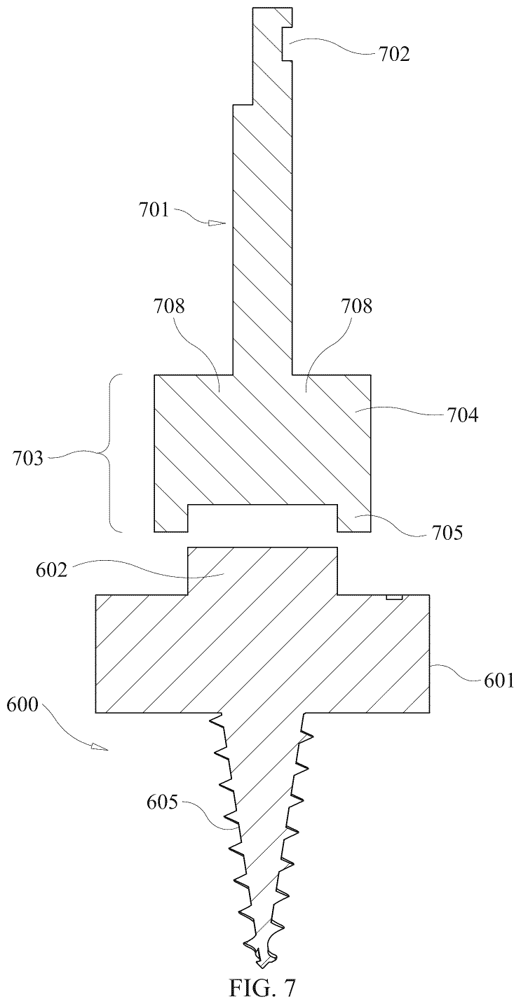

A driver 700 useful for placing a fiducial marker described herein is illustrated in FIG. 7. Driver 700 comprises a cylindrical shaft 701 having a first, drill engagement end 702 and a second, bolt-head engagement end 703. The first, drill engagement end of the driver shaft can be configured to detachably engage with a conventional variable-speed dental drill and drill handle, wherein the drill provides rotational force to turn the driver and an engaged screw head of the fiducial marker. The second, and distal end of the driver shaft, comprises a driver “head” 704 having a peripheral and cylindrical wall 705 extending to the distal end of the driver. The peripheral cylindrical wall is formed by a shoulder 208 jutting perpendicularly from the cylindrical shaft of the driver. The cylindrical wall has an outer face of about 4 mm in height.

At the distal end of the driver head, and within the confines of the cylindrical wall, is a recessed area 706 forming an inner face of the cylindrical wall, which is configured to matingly engage the peripheral sides of the fiducial marker bolt head.

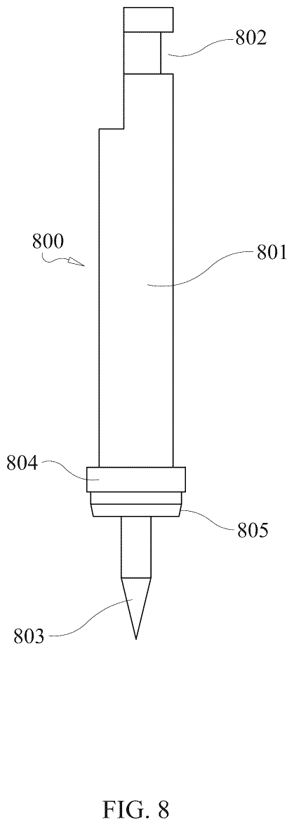

Also useful in conjunction with a fiducial marker of the subject invention is a drill bit, as illustrated in FIG. 8, to form a pilot hole into the bone as a guide for positioning the fiducial marker. A bone drill bit of the invention 800 comprises a cylindrical shaft 801 having a first, drill engagement end 802 and a second, drilling tip 803 for forming a pilot hole in bone. The first end of the drill bit shaft can be configured to detachably engage with a conventional variable-speed dental drill and drill handle, wherein the drill provides rotational force to turn the drill bit.

The second, distal end of the drill bit is formed as a pointed, apical tip to facilitate drilling into bone. Advantageously, the drill bit of the invention further comprises a circular, disc-shaped flange 804 affixed perpendicularly to the shaft, proximal to the second, distal drill end. The disc-shaped flange includes a sharp outer edge 805 on its distal face (facing the drilling end) to effect a circular cut or score into soft tissue overlying the bone and around the pilot hole. The disc-shaped flange, which can be referred to as a “tissue punch,” is about 2 mm in diameter, and is affixed to or preferably integrally formed with the drill bit shaft, and is positioned such that its leading edge is a specific, fixed distance, preferably about 3 mm, from the drill bit distal tip, thereby serving to indicate and gauge depth of the drill bit tip, i.e., identify when the tip is about 3 mm, into the bone. The circular cut or score formed in tissue overlying the bone can mark the area surrounding the pilot hole and facilitate identifying the location of the small pilot hole. In some instances, the cutting edge of the disc-shaped flange can be used to remove soft tissue, e.g., gum tissue, around the pilot hole, thus serving as a tissue punch.

The drill bit 800, fiducial marker 600, and driver 700, as described herein, can be used together as a system for placement of the fiducial marker in a dental procedure, e.g., performing a dental implant procedure. The system comprising the described drill bit, fiducial marker, and driver, can be employed as an aid in accurate diagnosis, treatment planning, and treatment outcome assessment.

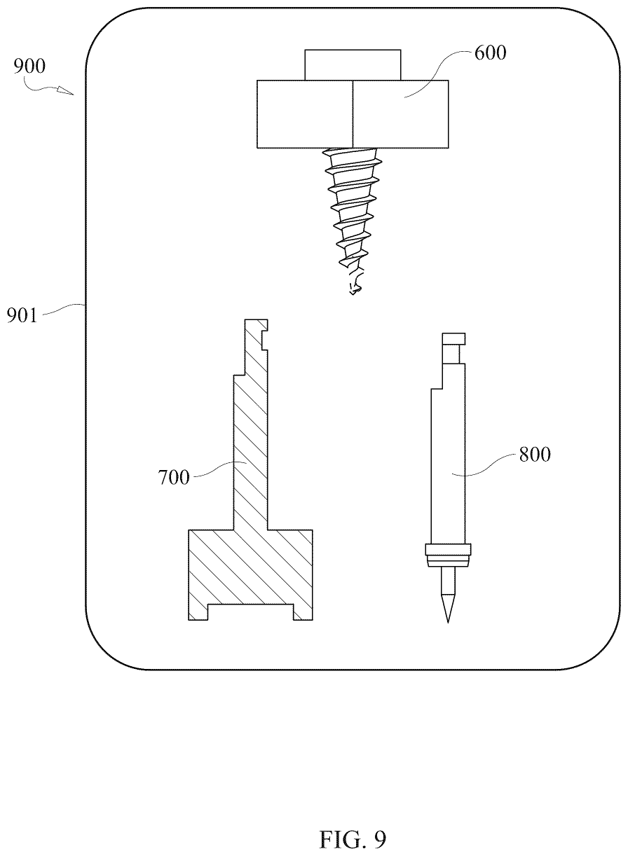

In accordance with the invention described, a kit can be provided, the kit comprising a drill bit 800, fiducial marker 600, and driver 700 for a fiducial marker of the invention, as shown in in FIG. 9. Specifically shown is a kit 900 enclosed within and packaged together, a fiducial marker of the invention 600, a driver 700 for a fiducial marker of the invention, and a drill bit 800 for drilling a pilot hole for guiding placement of a fiducial marker of the invention.

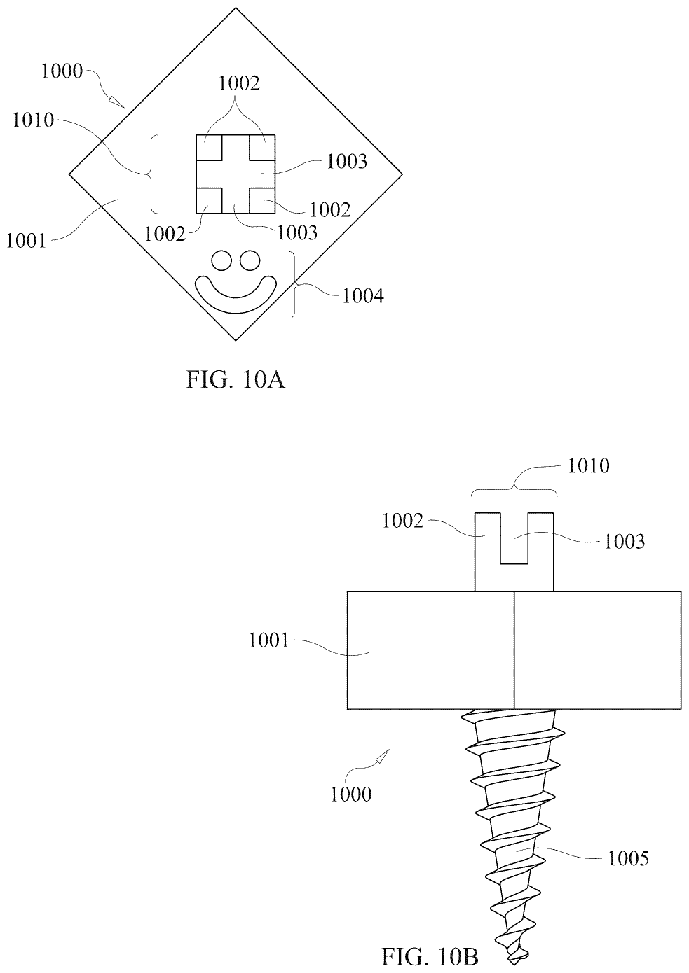

An alternative embodiment of the marker can comprise a slotted screw head instead of a flat bolt head. Referring to the drawings, FIG. 10A shows a top-view of an embodiment of a dental fiducial marker 1000 according to the subject invention, illustrating a square, flat flange base 1001 which is shown as being “diamond” shaped due to its orientation. The top face of flange base 1001 includes, integral therewith, a square screw head 1010, divided by slots 1003 in cross shape, forming four, square screw head bosses 1002.

The flat flange can be about 4 mm to about 8 mm in length on each of its sides, and can be about 1 mm to about 4 mm in thickness. In a preferred embodiment, the flat flange base is about 6 mm in length on each side ad about 3 mm in thickness.

On a top face of the flat flange 1001 is formed a slotted screw head 1010. Preferably, the slotted screw head 1010 extends about 2 mm above the top face of the flat flange base and is square, measuring about 1 mm to about 4 mm on each side. Preferably the screw head 1002 measures 2 mm×2 mm. The square screw head is orientated such that the sides are positioned at a 45-degree angle relative to the sides of the flat flange base 1001. Formed within the screw head 1010 are slots 1003 for receiving a driver for turning and placement of the fiducial marker.

In a preferred embodiment, the two slots are formed in the screw head at right angles to one another, forming cross-shaped slots. Due to their 45-degree angle orientation relative to the flange base, the slots are aligned such that they point to each of the four corners of the “diamond” shaped flange base. The slots are formed in the screw head as open slots, meaning that they extend to the edge of the screw head and are not closed or enclosed by a peripheral edge of the screw head. This open slot configuration results in four equal, and cubic screw head bosses 1002 forming screw head 1010.

The slots 1003 of the slotted screw head are formed to extend only partially into the screw head, wherein the slots have a depth of about 0.5 mm to about 1.5 mm, preferably a depth of about 1 mm. The slots are not formed to extend into the entire height of the screw head so that they are interconnected with one another and do not easily sheer from the flat flange base.

Also disposed on a top face of flat flange base 1001 is an indicia 1004, useful to ensure accurate and reproducible orientation of the fiducial marker. Indicia 1004 is shown in FIG. 10A as a “smiley face” configuration, but can be any identifier, including any identifying shape or design, number or numbers, letter or letters, or the like. Indicia 1004 can be radiopaque and comprises a single mark or identifier in one area on the top face of the fiducial flange base 1001, and more preferably, between one side of the screw head and a corner of the square or diamond-shaped, flat flange base. A preferred size of indicia covers an area having about 1 mm to about 2 mm radius.

On a bottom face of the flat flange base is disposed a screw shank, as illustrated in FIG. 10B. FIG. 10B depicts a side elevational view of an embodiment of a dental fiducial marker 1000 illustrated in FIG. 10A, showing the positional and spatial relation of the integral flat flange base 1001, screw head 1010, having slot 1003 formed therein to provide cubic screw head bosses 1002, and threaded, tapered (conical) screw shank 1005 extending from the bottom face of the flat flange base 1001. Threaded screw shank 1005 can be any needed length, typically from about 5 mm to about 8 mm in length, and is preferably about 6 mm in length. More preferably, threaded screw shank 1005 is conical and tapered to a point at its distal end to form a tap-screw configuration for ease of placement. A preferred width of the tapered, conical screw shank 1005 corresponds to the screw head 1010, and is preferably about 2 mm to about 3 mm in width at its widest dimension, at the juncture with the bottom face of flange base 1001, tapering down to a sharp point at the bottom end of conical screw shank 1005. Thread sizes (width, depth, and pitch) can vary and are accordance with conventional dental screws.

In use, a fiducial marker as described herein can be employed in a method for accurate diagnostics and treatment planning of a dental surgical procedure performed on a patient, said method comprising:
providing a dental fiducial marker as described and shown herein;
implanting said dental fiducial marker at a predetermined position in the patient's mouth or dental arch; and
imaging the patient's mouth or dental arch to assess position, alignment, and spatial relationships of dental structures in the patient.

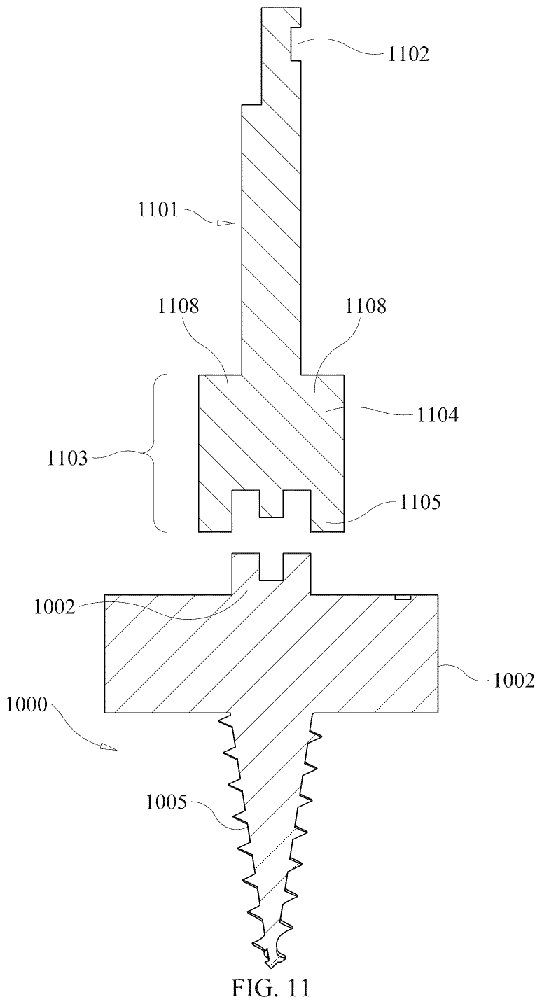

A driver 1100 useful for placing a fiducial marker having a slotted screw head as described herein is illustrated in FIG. 11. Driver 1100 comprises a cylindrical shaft 1101 having a first, drill engagement end 1102 and a second, screw-head engagement end 1103. The first, drill engagement end of the driver shaft can be configured to detachably engage with a conventional variable-speed dental drill and drill handle, wherein the drill provides rotational force to turn the driver and an engaged screw head of the fiducial marker. The second, and distal end of the driver shaft, comprises a driver “head” 1104 having a peripheral and cylindrical wall 1105 extending to the distal end of the driver. The peripheral cylindrical wall is formed by a shoulder 1108 jutting perpendicularly from the cylindrical shaft of the driver. The cylindrical wall has an outer face of about 4 mm in height.

At the distal end of the driver head for a marker having a slotted screw head, and within the confines of the cylindrical wall, is a recessed area 1106 forming an inner face of the cylindrical wall, which is configured to matingly engage the peripheral sides of the fiducial marker screw head. Formed within the recessed area is a cross-shaped embossed driver blade 1107 for matingly engaging the cross-slotted screw head the fiducial marker of the invention. The driver blade is preferably configured to have a height which is less than the height of the outer cylindrical wall, typically about 1 mm to about 1.5 mm in height.

Also useful in conjunction with a fiducial marker of the subject invention is a drill bit, as illustrated in FIG. 8, to form a pilot hole into the bone as a guide for positioning the fiducial marker.

The drill bit, fiducial marker, and driver, as described herein, can be used together as a system for placement of the fiducial marker having a slotted screw head in a dental procedure, e.g., performing a dental implant procedure. The system comprising the described drill bit, fiducial marker, and driver for a marker having a slotted screw head, can be employed as an aid in accurate diagnosis, treatment planning, and treatment outcome assessment.

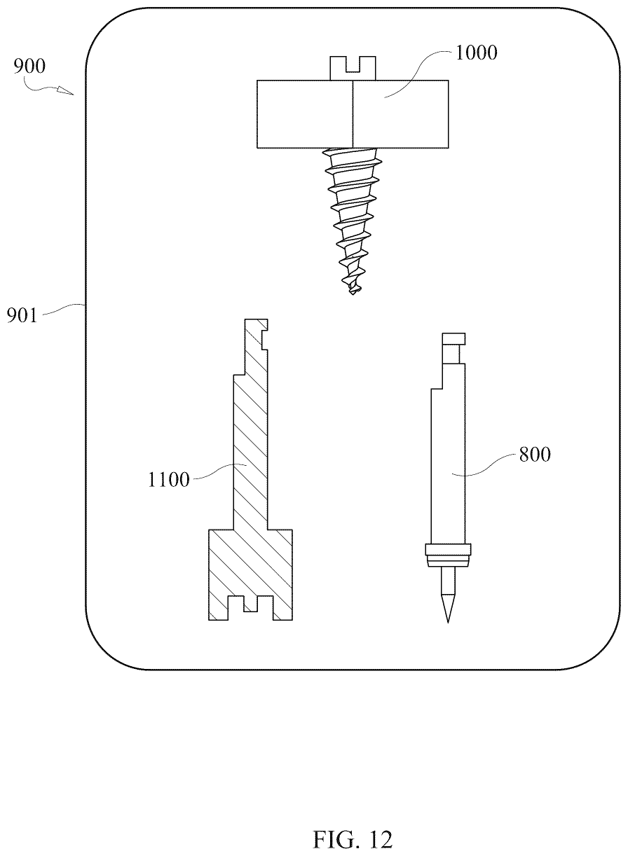

In accordance with the invention described, a kit can be provided, the kit comprising a drill bit, fiducial marker, and driver for a fiducial marker of the invention, as shown in in FIG. 12. Specifically shown is a kit 1200 enclosed within and packaged together, a fiducial marker of the invention 1000, a driver 1100 for a fiducial marker of the invention having a slotted screw head, and a drill bit 800 for drilling a pilot hole for guiding placement of a fiducial marker of the invention.

The foregoing description is illustrative only and is not intended to limit the scope of the invention to the precise terms set forth. Although the invention has been described in detail with reference to certain embodiments, variations and modifications exist within the scope and spirit of the invention as described and defined in the following claims. The above disclosure and examples generally describe the present invention and are provided for purposes of illustration and is not intended to limit the scope of the invention. The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. An implant fixture configured as an elongated post forming a body of the implant fixture having a distal end, a proximal end, and an outer side surface, said side surface being partially threaded on a portion of the outer side surface and having a smooth or polished portion of the outer side surface.

2. The implant fixture of claim 1, wherein the proximal end of the implant fixture body is tapered to form an apical end.

3. The implant fixture of claim 2, wherein the apical proximal end comprises threads completely encircling the implant fixture body.

4. The implant fixture of claim 3, wherein the threads completely encircle the implant fixture body from the apical end to about 3-5 mm from the apical end.

5. The implant fixture of claim 1 wherein the distal end of implant fixture body comprises a recess formed therein for matingly receiving a drill bit for tightening or loosening the implant fixture.

6. The implant fixture of claim 1 wherein the polished or smooth outer side surface extends from the distal end to the apical end comprising completely encircling threads.

7. The implant fixture of claim 1, wherein the threads of the partially threaded outer surface extend about 50% to about 99% around the implant fixture body.

8. The implant fixture of claim 1, wherein the implant fixture body is titanium or titanium alloy.

9. The implant fixture of claim 1, wherein the threaded portion of the implant fixture body can be coated.

10. A dental implant system comprising an implant fixture of claim 1, and comprising an abutment affixable to the implant fixture, a connecting screw, and prosthetic tooth affixable to the abutment.

11. The dental implant system of claim 10, optionally comprising a healing cap for temporary placement over an implant fixture.

12. The dental implant system of claim 10, wherein the implant system is provided for performing a Palatal Approach technique or whenever a patient has a lingual or buccal concavity defect.

13. A method for implanting a prosthetic tooth or plurality of teeth in a patient in need thereof, said method comprising the steps of:
a) providing an implant fixture of claim 1;
placing the implant fixture within the jawbone of the patient at a desired depth such that the fully threaded surface is oriented within the bone and the polished or smooth surface is oriented to be at least partially exposed outside the bone;
c) affixing an abutment to the implant fixture; and
d) affixing a prosthetic tooth or plurality of teeth to the abutment.

14. The method of claim 13, further comprising the step:
temporarily affixing a healing cap to the implant fixture prior to affixing the abutment to the implant fixture.

15. The method of claim 13, further comprising the step of:
surgically placing or replacing periodontal tissue over the exposed polished or smooth face of the implant fixture, without bone reconstruction.

16. The method of claim 13, wherein the steps are carried out using a Palatal Approach technique.

17. A unitary dental fiducial marker comprising:
a flat, geometrically shaped flange base having a top face and a bottom face;
a threaded conical screw shank or screw body extending from the bottom face of the flat flange base;
an unslotted bolt head or slotted screw head disposed on the top face of the flange base to receive a driver; and
at least one indicia for positional recognition of placement and orientation during an imaging procedure;
wherein the bolt head or screw head, flange base, and threaded conical screw shank are integrally formed to provide the unitary dental fiducial marker.

18. The unitary dental fiducial marker of claim 17, wherein the at least one indicia is disposed on the top face of the flange base.

19. The unitary dental fiducial marker of claim 17, wherein the marker comprises a bolt head having a flat top face, and the at least one indicia is disposed on the flat top face of the bolt head.

20. The unitary dental fiducial marker of claim 17, wherein the marker comprises a slotted screw head and said slotted screw head comprises two slots in the top face of the screw head, said two slots being centered and perpendicular to one another, forming a cross-shaped slotted screw head.

21. The unitary dental fiducial marker of claim 20, wherein the slotted screw head provides four equal cubic bosses.

22. The unitary dental fiducial marker of claim 20, wherein the slotted screw head has a height of about 2 mm above the flat, top face of the flange base.

23. The unitary dental fiducial marker of claim 20, wherein the two cross slots have a depth of about 1 mm to about 1.5 mm.

24. The unitary dental fiducial marker of claim 17, wherein the indicia is radiopaque.

25. The unitary dental fiducial marker of claim 18, wherein the indicia is configured as a smiley face symbol.

26. The unitary dental fiducial marker of claim 17, wherein the flat flange base is square wherein each side is about 6 mm in length.

27. The unitary dental fiducial marker of claim 17, wherein the flat flange base has a thickness of about 1 mm to about 3 mm.

28. The unitary dental fiducial marker of claim 17, wherein the threaded screw shank or screw body is conical and tapered and configured as a tap screw.

29. The unitary dental fiducial marker of claim 17, wherein the threaded screw shank has a length of about 5 mm to about 8 mm.

30. A method for accurate diagnostics and treatment planning of a dental surgical procedure performed on a patient, said method comprising:
providing a dental fiducial marker of claim 17;
implanting said dental fiducial marker at a predetermined position in the patient's mouth or dental arch; and
imaging the patient's mouth or dental arch to assess position, alignment, and relationships of dental structures in the patient.

31. A driver for a fiducial marker, said driver comprising a cylindrical shaft having a first end configured to detachably engage with a conventional variable-speed dental drill and drill handle, and a second, distal end comprising a driver head configured to matingly engage a bolt head or screw head and flange base of a fiducial marker of claim 17.

32. A drill bit for drilling a pilot hole for guiding placement of a fiducial marker, said drill bit comprising a cylindrical shaft having a first end configured to detachably engage with a conventional variable-speed dental drill and drill handle, and a second, distal end having a drill bit for drilling a pilot hole for receiving a fiducial marker of claim 17, said drill bit further comprising a disc-shaped flange affixed perpendicular to the cylindrical shaft, said disc-shaped flange comprising a sharp peripheral edge on its distal face for cutting or scoring tissue overlying bone and marking the pilot hole formed by the drill bit.

33. A kit comprising a fiducial marker of claim 17, a driver for use with said fiducial marker, and a drill bit comprising disc-shaped flange comprising a sharp peripheral edge on its distal face for cutting tissue overlying bone.
